(19) United States
(12) Patent Application Publication
(10) Pub. No.: US 2013/0226022 A1
(43) Pub. Date: Aug. 29, 2013

(54) Measurement Device for Lung Function Measurement

(71) Applicants: Roland Erwin John Marges, Baarn (NL); Sander Willem Van Schaik, Utrecht (NL); Gerard Rudolf Riemens, Mierlo (NL)

(72) Inventors: Roland Erwin John Marges, Baarn (NL); Sander Willem Van Schaik, Utrecht (NL); Gerard Rudolf Riemens, Mierlo (NL)

(73) Assignee: CCM Beheer B.V., Nuenen (NL)

(21) Appl. No.: 13/821,501

(22) PCT Filed: Sep. 12, 2011
(86) PCT No.: PCT/EP2011/065728

§ 371 Date: May 13, 2013

Related U.S. Application Data
(60) Provisional application No. 61/381,456, filed on Sep. 10, 2010.

(51) Int. Cl. A61B 5/085; A61B 5/087

(52) U.S. Cl. CPC A61B 5/085; A61B 5/087
USPC 600/538

(57) ABSTRACT

The invention comprises a measurement device for lung function measurement comprising a flow tube forming an air-flow path, an actuator for closing and opening the flow tube and at least one sensor for measuring at least one lung function variable, wherein the actuator is arranged to force the flow tube to close the air-flow path by pressing the tube against a wall of the flow tube.

The present invention relates to an occlusion technique for lung function measurement and more in particular the invention relates to a measurement device for lung function measurement

To measure lung function, various techniques have been developed in the past, including the Single Occlusion Technique (SOT) and the Resistance by interruption (Rint) technique. These techniques make use of a temporarily obstruction (occlusion) in the air-flow path to interrupt a normal breathing cycle for a short period of time. During this occlusion, measurement of specific variables such as pressure and flow is performed. Air-way resistance can then by calculated out of flow and pressure.

More specific, the Single Occlusion Technique (SOT) makes use of the Hering-Breuer reflex of neonates and small children up to 12 months of age. This technique is thus for neonates up to 1 year. The Rint technique is suitable for humans in an age range up to 100 years. This technique calculates the internal resistance of the airways and is used for people who are not capable to produce peak flows or follow instructions of the care taker. The Spirometry technique is suitable for humans in an age range of about 5-100 years. This technique measures peak flows and volumes during forced deep exhalation and inhalation periods.

For the SOT/Rint measurements three values are necessary to calculate the requested respiratory parameters: air-flow, volume and pressure. For the Spirometry variables flow and volume values are necessary for calculating the respiratory parameters.

The flow is measured with three ultrasonic transducers with the time of flight principle; one transducer acts as sender and emits an ultrasonic wave towards the other two receiving transducers. The time difference between the receiving transducers receiving the ultrasonic wave is a measure for the air-flow traveling between the transducers. The volume is calculated by integrating the flow over time. During an occlusion the air-flow is blocked and depending on the moment during a breath cycle of occlusion, pressure is build up in the flow tube. This pressure should be measured as close as possible near the mouth. The way the respiratory parameters are calculated have been published by the European Respiratory Society and the American Thorac Society.

The MicroRint distributed by CareFusion uses an occlusion technique for Rint measurements. In practice, it is not possible to measure the compliance of babies between 0-1 years old since it does not use the Hering-Breuer reflex of the baby and it is a noisy measurement resulting in awakening sleeping babies. Occlusion is realized by a “butterfly valve”. This construction is not persistent against lower pressures.

Exhalyzer D distributed by ECO MEDICS has a system which is used for measurements of neonates and non-cooperative children in a clinical environment. This system is not handheld.

It is an object of the present invention, among other objects, to improve upon the known measurement devices and/or to provide a compact, efficient and/or reliable measurement device for lung function measurement.

This object, among other objects, is met by a measurement device according to claim 1.

More in particular, this object, among other objects, is met by a measurement device for lung function measurement comprising a flow tube forming an air-flow path, an actuator for closing and opening the flow tube and at least one sensor for measuring at least one lung function variable, wherein the actuator is arranged to force the flow tube to close the air-flow path by pressing the tube against a wall of the flow tube. By closing the air-flow path in the flow tube using an actuator extending outside the flow tube, contamination of the occlusion actuator and of the inner surface forming said air-flow path is prevented, while obtaining an efficient closure of said air-flow path allowing measurement of lung functions using at least one sensor, for instance in the form of a transducer.

Preferably the flow tube comprises a plurality of sensors, for instance transducers. More preferably, the sensor is arranged to measure at least one of air-flow, volume and pressure in the air-flow path of the flow tube.

The interior of the flow tube or tubular member of the measurement device forms an air-flow path for the air to be measured. The measurement device is preferably provided with an air inlet, for instance in the form of a mouth piece, in fluid connection with said flow tube.

The actuator is preferably arranged to press two preferably substantially diametrically opposed wall parts of the flow tube together for closing the air-flow path. At least in the region of the working area of the actuator the flow tube is therefore manufactured from a flexible, in particular elastic, material allowing the pressing together of the walls of tube. It is hereby possible that another part of the flow-tube, outside the working area of the actuator, is manufactured from a stiff material.

The actuator is preferably moveable between a closing position, wherein the air-flow path is closed, and a resting position, wherein the flow tube is substantially non-deformed by the actuator. In the closing position, the actuator extends within the area of the inner circumference of flow-tube in the non-deformed state. More preferably the actuator extends within the inner circumference along a distance at least equal to the inner diameter of the flow-tube in the non-deformed state. In the resting position, the actuator preferably extends outside the outer circumference of the flow-tube in non-deformed state. The actuator is preferably moveable in a direction with a component perpendicular to the longitudinal axis of the flow tube or parallel to a normal of the wall of the flow tube in non-deformed state.

Preferably the flow tube has elastic characteristics for opening the air-flow path upon reversal of the actuator. The flow tube is preferably manufactured from a resilient or elastic material, at least in the working area of the actuator. This results in an efficient, passive opening of the air-flow path upon withdrawal, i.e. movement to the resting position, of the actuator. More preferably the flow tube comprises a silicon rubber tube.

According to a preferred embodiment of the measurement device according to the invention flow tube is provided with two cut-outs in the contour. The cut-outs provided in the outer surface of the flow tube allow an efficient closure of the flow tube. Less force is needed to press the walls of the tube together along the whole inner circumference of the flow tube. The cut-outs may be provided along at least a length in the working area of the actuator, it is however also possible that the cut-outs extend along substantially the whole length of the flow tube. Preferably the cut-outs are provided at diametrically opposed locations in the contour or outer surface.

According to a further preferred embodiment the flow tube or the measurement device comprises a stiff wall, wherein the actuator is arranged to force the flow tube to close the air-flow path by pressing the tube against the stiff wall. Said stiff wall is preferably arranged stationary in the measurement device, for instance as a housing, wherein the actuator is moveable to and from the wall and wherein the flow tube extends between the stiff wall and the actuator.

Preferably the wall comprises a U-shaped groove arranged to receive the flow tube and/or the actuator in the closed position. In the closing position, the flow tube is forced in the U-shaped groove by the actuator, thereby enhancing the closing action.

According to a further preferred embodiment of a measurement device according to the invention the actuator is provided with V-shaped end. Preferably, the V-shaped end is arranged to be received in the groove of the wall together with the walls of the flow tube for closing the air-flow path. The V-shaped end allows a more local deformation of the flow tube, thereby further enhancing the closing action.

According to a further preferred embodiment the actuator is biased towards the open air-flow path position or resting position. The actuator will thereby always return to the open position upon disengagement of the actuator. Preferably, the actuator is provided with resilient means, for instance a spring, for automatically or passively urging the actuator to the open position.

It is possible to form the flow tube as a disposable element. However, according to a preferred embodiment the sensor, for instance in the form of an ultrasonic transducer, is formed integrally and water-tight with the flow tube. This allows easy cleaning of the flow tube and therefore reuse of the flow tube. Preferably the sensor is hereto encapsulated in casing filled with a sealant, preferably silicon rubber sealant.

A preferred embodiment of a measurement device according to the invention is formed as a handheld. This makes the measurement device according to the invention in particular suitable for use on neonate and small children (<1 years).

A preferred embodiment of a measurement device according to the invention comprises a cradle and a flow tube part as separate bodies, wherein the cradle is arranged to interchangeably receive the flow tube part and wherein the actuator is arranged in the cradle. This allows the flow tube part and in particular the flow tube thereof, which is the part in contact with the patient, to be removed from the cradle. The flow tube (part) can be cleaned or discarded after use.

Preferably the flow tube is provided with at least one sensor, for instance in the form of a transducer, and wherein the cradle and the flow tube part are provided with cooperating electric spring contacts. The electrics signals from the sensor in the flow tube are provided to suitable processing means in the cradle.

More preferably the cradle further comprises at least one of a pressure sensor, a battery and digital electronics. The pressure sensor can be operable connected to sensing means in the flow tube, for instance in the form of a fluid connection between the pressure sensor and the air-flow path of the flow tube.

According to a preferred embodiment, the flow tube, or flow tube part, is arranged for SOT/Rint-measurements, wherein a pressure sensor, or sensing means as described above, is provided near the inlet of the flow tube, more preferably near a mouth piece, for sensing the pressure near said inlet.

It is however also possible to provide a flow tube, or flow tube part, for spirometry-measurements. The invention furthermore relates to a kit of parts comprising a cradle, a flow tube part for SOT/Rint-measurements and a flow tube part for spirometry-measurements, wherein the cradle and the flow tubes parts are arranged to be interchangeably connected.

The invention furthermore relates to a cradle and a flow tube part for use in a measurement device according to the invention.

The present invention is further illustrated by the following Figures, which show a preferred embodiment of the device according to the invention, and are not intended to limit the scope of the invention in any way, wherein is shown:

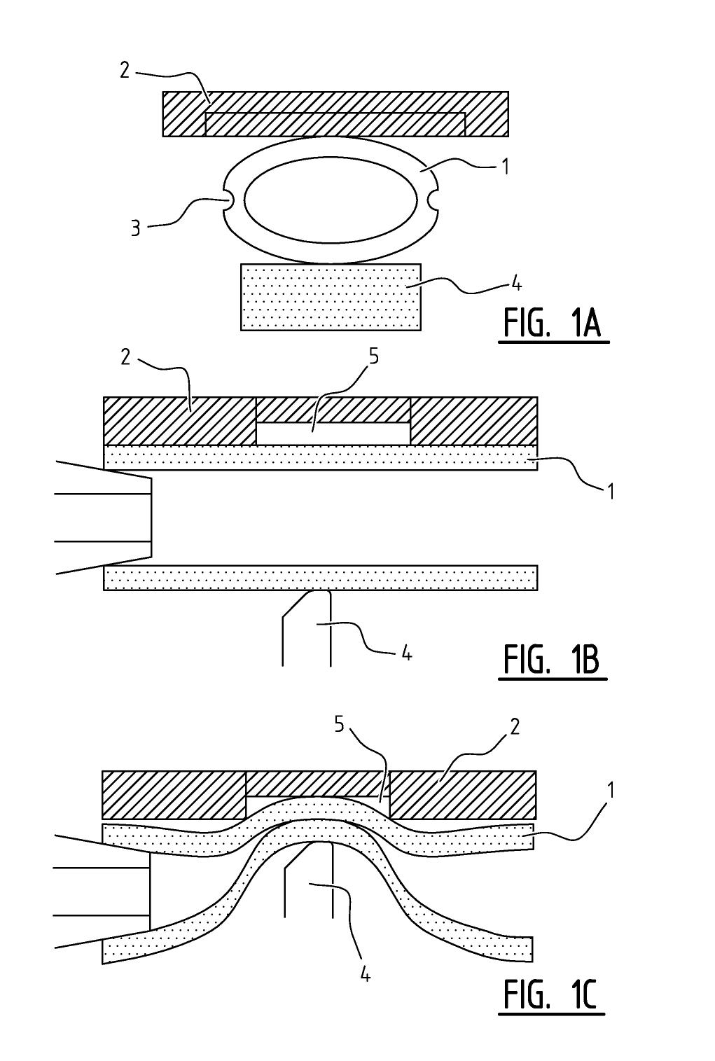

FIG. 1A: Shutter in open situation front view.

FIG. 1B: Shutter in open situation side view.

FIG. 1C: Shutter in closed situation side view.

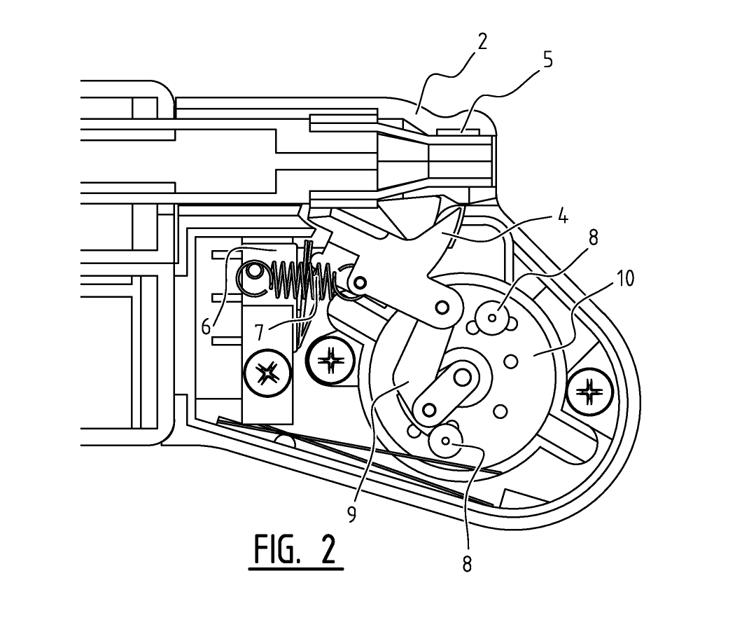

FIG. 2: Shutter actuator.

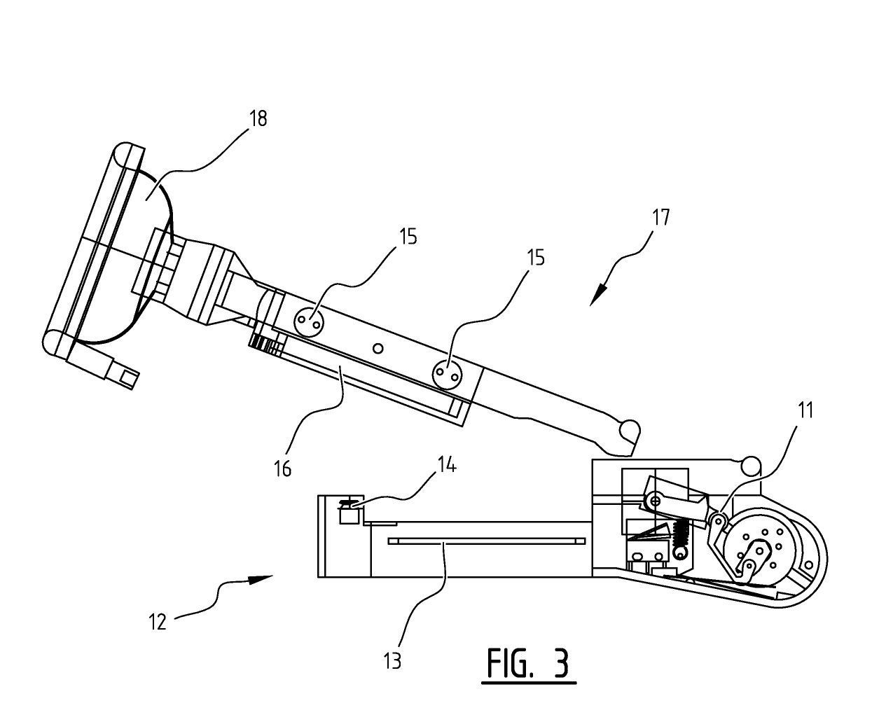

FIG. 3: Cradle and SOT/Rint mouthpiece.

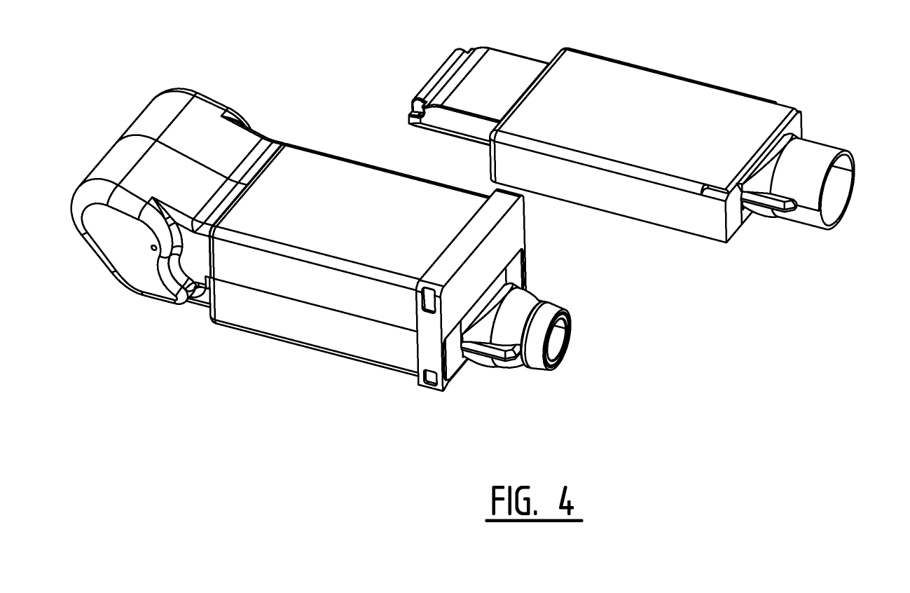

FIG. 4: Cradle and SOT/Rint- and Spirometry mouthpiece.

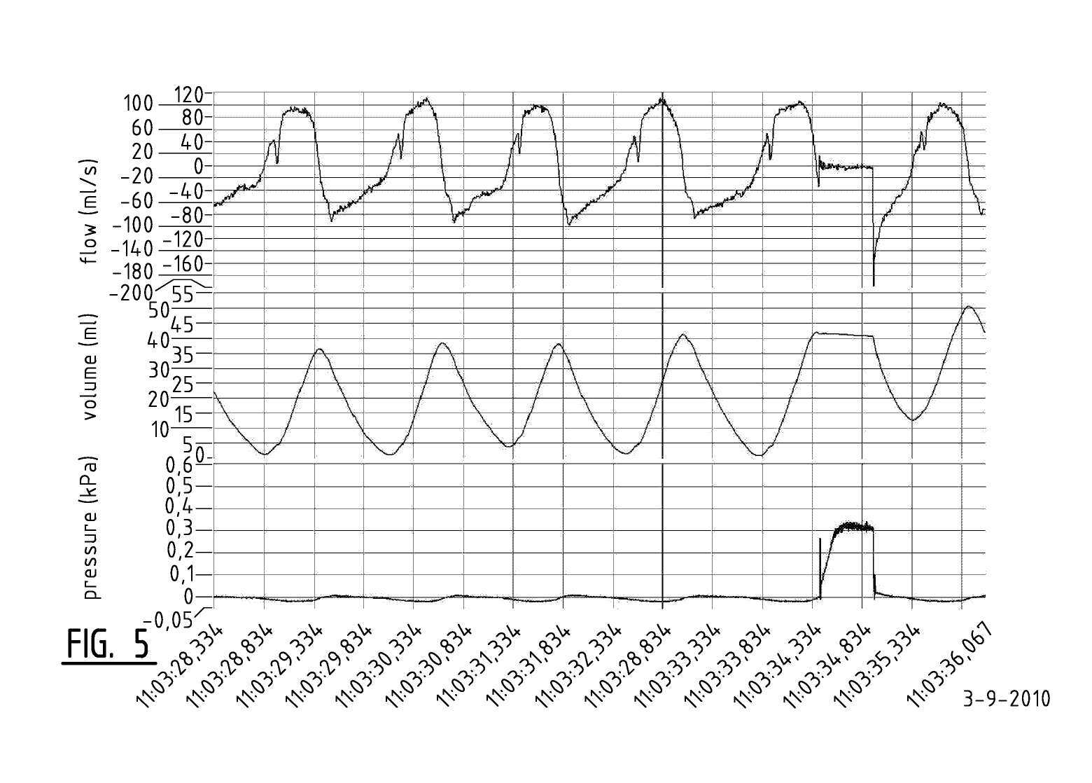

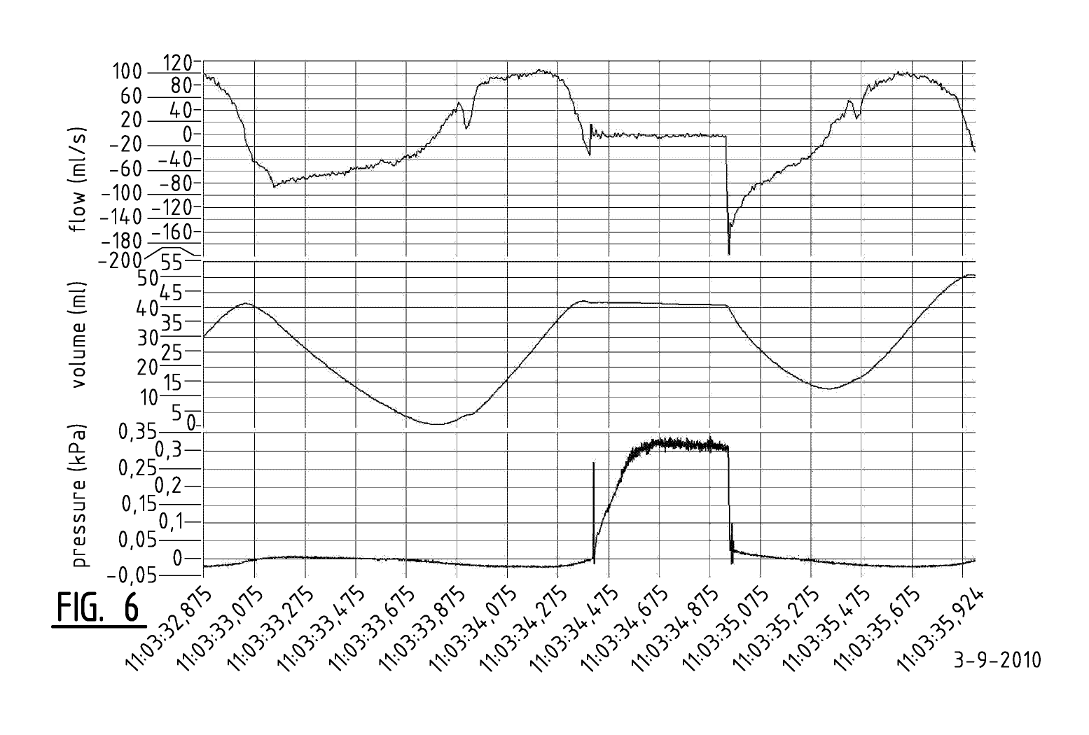

FIGS. 5 and 6 show results of a SOT-measurement in graphs containing raw unfiltered data.

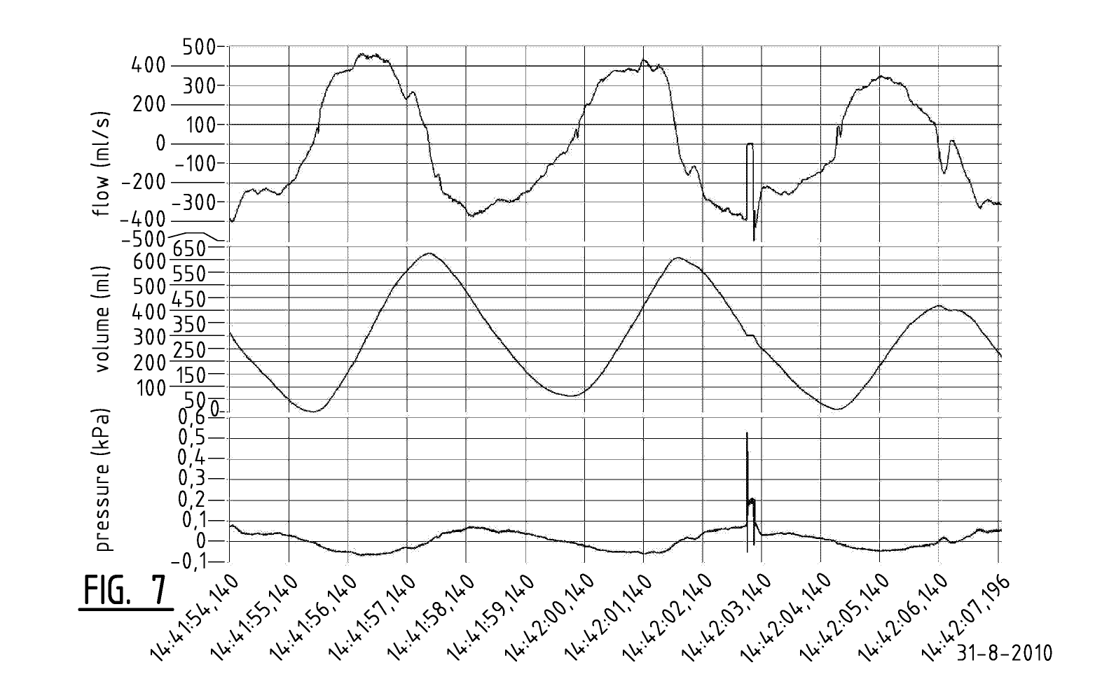

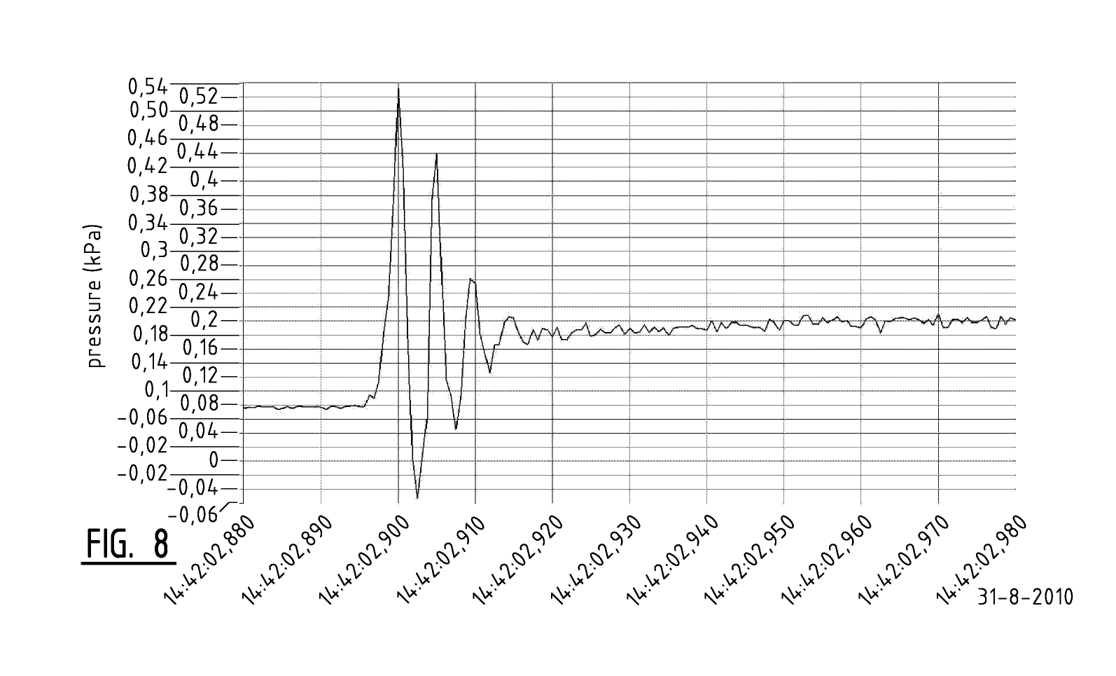

FIGS. 7 and 8 show results of a RINT-measurement in graphs containing raw unfiltered data.

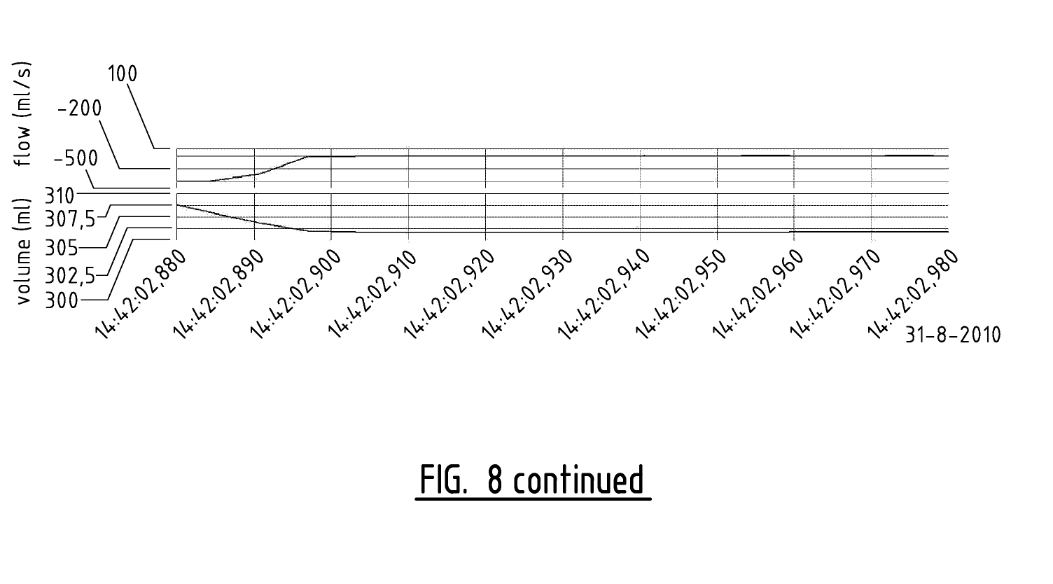

FIGS. 9a and 9b schematically show a further embodiment of the measurement device according to the invention in connected, respectively disconnected state.

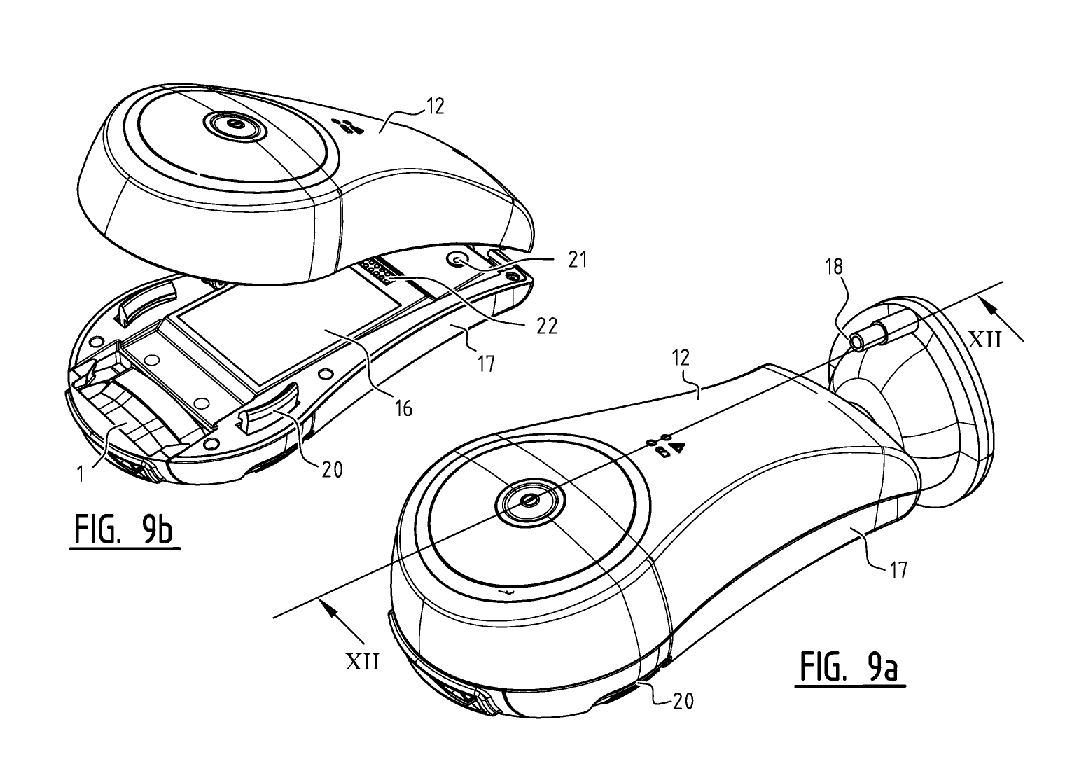

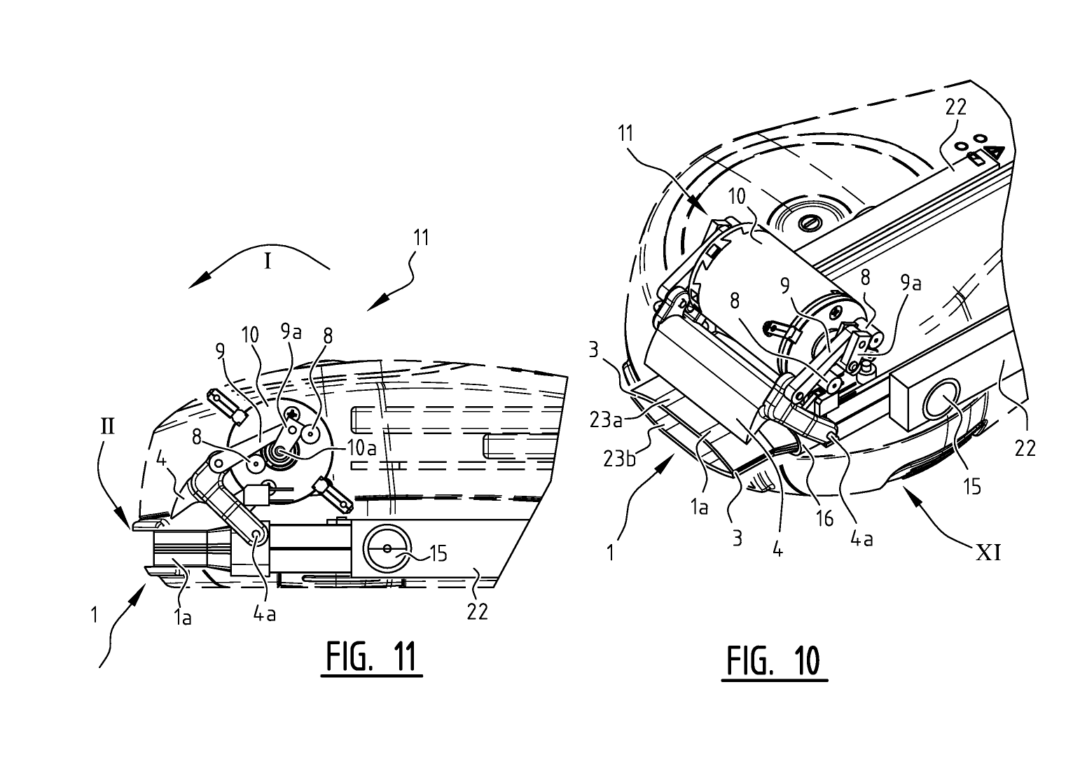

FIGS. 10 and 11 schematically shows the shutter mechanism in perspective, respectively in side view.

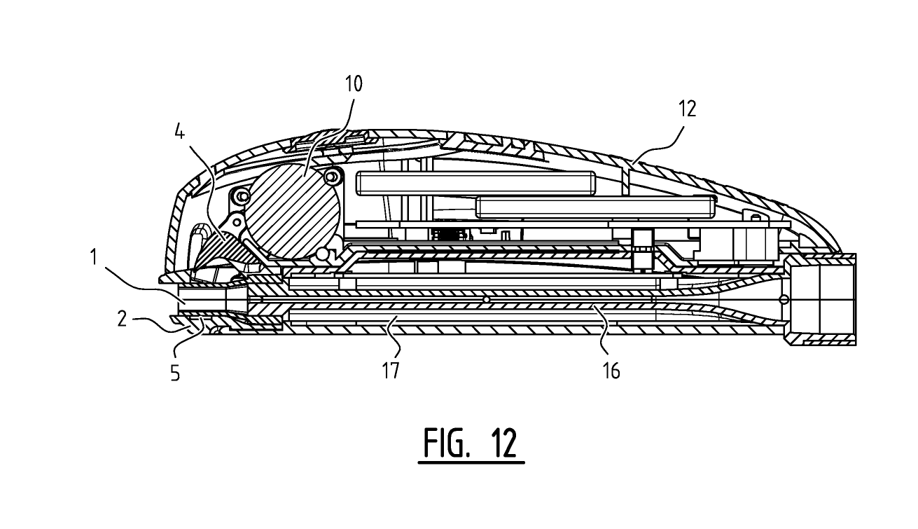

FIG. 12 schematically shows the measurement device in cross-section.

To realize occlusion, a shutter (called a shutter knive) is integrated in the SOT/Rint flow tube, see FIGS. 1a-c and FIG. 2, in order to minimize dead space. The shutter 4 is closed and opened by an actuator 9, see FIG. 2. The actuator consists of an electrical device 10 for delivering the necessary force and mechanics to transport the delivered force onto the shutter. The shutters' actuator mechanism 9 has no physical contact with the inside of the flow path and can't be contaminated. The shutter, functioning as a valve, is constructed out of a soft silicon rubber tube 1 or other elastic/flexible material. This silicon rubber tube has been shaped with two cut-outs 3 in the contour, see FIG. 1A. The shutter actuator forces the silicon rubber tube 1 to close the air-flow path by pressing the tube against the stiff wall 2 of the flow tube. In this wall 2 a small u-shaped groove 5, see FIG. 1B, has been made which with the shutters' actuator 4 specific V-shape provide for an airtight closing of the flow path. This total occlusion construction prevents a pressure surge which disturbs the pressure measurements.

For opening the flow path the shutters' actuator is reversed and the shutter is opened by its own elastic characteristics. A detection of the position of the shutters' actuator by an open/close sensor 6 is incorporated for safety purposes. Further, a spring 7 has been integrated to create a failsafe situation if the shutters' actuator becomes de-energized. The spring 7 will position the shutters' actuator in the “open air-flow path” position. Because the shutter itself is an external mechanism that is not coming into contact with the flow path of the patient, this is a hygienic occlusion solution. Contamination of the shutter can therefore never influence the quality and cleanness of the flow tube.

The construction is designed for noise suppression when the shutter is opened or closed by low mass of the moving parts and rubber damping, indicated with 8 in FIG. 2, of the moving parts.

### ADVANTAGES OF THE INVENTION

The above described occlusion technique has the following advantages:
Low energy use for closing the shutter.
Hygienic occlusion solution.
Minimal pressure surges in the flowtube when closing or opening the shutter.
Minimal dead volume added to the system.
Low noise operation (to prevent sleeping babies to awake).

In one embodiment, the above described occlusion technique will be used in an all-in-one device that allows lung function measurement, irrespective of the age of the human individual, by integrating three different technical types of measurement (SOT, Rint, Spirometry). Such device will consist of a cradle 12 and two separate flow tubes or flow tube parts: one as indicated with 17 in FIG. 3 for SOT and Rint measurements, the other for Spirometry measurement. The Spirometry flow tube with less internal resistance was developed in order to handle large peak flows. The instrument is able to measure air-flow, air-volume and pressure, i.e. an all-in-one measurement device.

Such devices may include flow tubes that contain 1, 2, 3 or more Ultra Sonic transducers 15 for measurement of air-flow and air-volume and the electronics 16 for actuating the transducers 15 and processing signals of the transducers 15.

Only the flow tube or flow tube part 17 for SOT/Rint has a connection for measuring pressure in the flow-tube as near as possible to the mouth piece. The internal volume of the SOT/Rint flow-tube has been minimized to be able to measure babies and even neonates. The dead volume of the flow tube for SOT/Rint is less than 11 ml. To minimize the ultrasonic reflections inside the flow tube a special texture of ribs has been developed and realized.

The cradle 12 contains the pressure sensor 14, the shutters' actuator/occlusion mechanism 11, the rechargeable battery and the digital electronics 13. The digital electronics 13 consists of a micro controller, with memory, a blue tooth connection and interfacing with all other electronics in- and outputs of the device. The electrical connection between cradle 12 and flow tube 17, in particular the flow tube part, is established by electrical spring contacts.

The flow tubes or flow tube parts can be constructed as a disposable component, thus maximizing patient hygiene, or as a re-usable component. The latter should be cleaned after each measurement. Also, since electronics 16 and US transducers 15 are integrated in the flow tube parts 17, the flow tube part is made watertight for protection of the electronics 16 and transducers 15 during the cleaning process. Preferably, the seams of the tube are prepared to avoid entering moisture into the compartment in which the electronics 16 is enclosed. Also, the US transducers 15 are preferably encapsulated in an aluminum case and filled with a silicon rubber sealant. The sensors, for instance in the form of transducers, are preferably integrated in the flow tube in a way that the flow tube can be cleaned inside and outside without obstacles in the flow path.

For the functioning of the described device, as well as, but not limited to, functioning of alternative devices described below, we have developed dedicated firmware. The firmware in the device cooperates with the specially developed software for a personal computer (PC) by means of a wireless connection (e.g. Blue tooth). This connection is automatically established when the device is switched on. It also enables automatic recognition of the type of flow tube that is used. Special attention is given to minimize communication delays for measurements and to optimize the quality of the test result. The software in the PC shows the operator a User Interface (UI) with which a choice can be made for the type of measurement and for the starting a measurement. During a measurement, the values are continuously presented to the user on the screen by means of a graph. When conducting a SOT or Rint measurement, the software activates an occlusion at the proper moment of breathing. The software contains the necessary algorithms to determine the proper sequence of breathing after which an occlusion may occur. With the collected data, the software determines the requested respiratory parameters for every type of measurement. These respiratory parameters are summerized in a table:

| SOT | Rint | Spiro |
|---|---|---|
| Peep [kPa] | Rrs [kPa * s/l] | FVC [l] |
| Vnul [ml] | Flow [ml/s] | FEV1 [l] |
| Vmax [ml] | Pint [kPa] | FEV1/FVC |
| Vplat [ml] | P(to) [kPa] | PEF [l/s] |
| sd Vplat [ml] | Ppre [kPa] | FEF25-75 [l/s] |
| rPplat [kPa] | Ppost [kPa] | FEF25 [l/s] |
| sd Pplat [kPa] | | FEF50 [l/s] |
| Length [s] | | FEF75 [l/s] |
| Crs [ml/kPa] | | FET [s] |
| r-VQ | | EV [l] |
| Vbeg [%] | | FEV6 [l] |
| Vend [%] | | (FIVC [l]) |
| Rrs [kPa * s/l] | | (PIF [l/s]) |
| Trs [s] | | |

Table 1 respiratory parameters measured with the instrument.
The described device has the following advantages:

One product for three different measurement techniques;
Measuring lung functions of babies and young children;
One product for broad range of air-flows, pressures, resistances;
Different flow tube parts in combination with one base device;
Automatic shutter (shuts at the right time in the breathing process);
Contamination of the shutter mechanism is not influencing the quality/cleanness of the flow tube;
Cleanable sensors;
SOT is unique for a handheld device;
Rint is in our device easy applicable;
Low dead volume;
All-in-one device for lung function measurement for total population (0-100 years).

In other embodiments, the above described occlusion technique will be used in alternative devices that measure only SOT or Rint, or a combination of SOT and Rint. Such devices may include flow tubes that contain 1, 2, 3, or more Ultra Sonic transducers for measurement of air-flow and air-volume and the electronics for actuating the transducers and processing the signals of the transducers.

### EXAMPLES

A demonstrator, as shown in FIG. 4, is available with one flow tube for SOT/Rint measurements and one flow tube for Spirometry measurements.

First measurements have been done for SOT on sleeping babies and Rint on young children. The acquired values with the demonstrator are subject to comparing with reference measurements on the same object. These reference measurements are done with the SOT equipment developed by UMC-Utrecht and by a MicroRint. The results are described below with reference to FIGS. 5-8.

Measurement Results

SOT-Measurement

| Patient Information: | |
|---|---|
| Name | N N |
| Sex | F |
| Date of birth | 24/07/2010 |
| Age | 5 weeks |
| Weight | 5 kg |
| Height | 0.58 cm |
| Measurement | |
| Information: | |
| Start of measurement | 2010/09/03 10:57:14 |
| Type of measurement | SOT (Single Occlusion Technique) |
| Datafile | 100903_SOT_2438_20100903_105714.tdms |
| Software-version | v1.11 |
| Cradle | CCMPROTO-10060001 |
| Sensor Used | TSRP-10030001 |
| Sensor Calibration Date | 2010/04/06 |

FIG. 5 shows the results of the SOT-measurement: automatic occlusion of 500 ms after 5 comparable tidal breathings.

FIG. 6 shows the results of the SOT-measurement: zoomed in on the occlusion.

Calculated result for this occlusion:

| | |
|---|---|
| Occlusion | 11:03:34.40 |
| EE-cursor | 11:03:33.79 |
| Peep [kPa] | 0.0030 |
| Vnul [ml] | 0.0000 |
| Vmax [ml] | 41.4581 |
| Vplat [ml] | 40.4422 |
| sd Vplat [ml] | 0.0535 |
| rPplat [kPa] | 0.3178 |
| sd Pplat [kPa] | 0.0077 |
| Length [s] | 0.3188 |
| Crs [ml/kPa] | 109.8719 |
| r-VQ | 0.9917 |
| Vbeg [%] | 82.0000 |
| Vend [%] | 32.0000 |
| Rrs [kPa * s/l] | 2.1221 |
| Trs [s] | 0.2331 |

Rint-Measurement

| Patient Information: | |
|---|---|
| Name | N S |
| Sex | M |
| Date of birth | 05/04/2002 |
| Age | 8 years |
| Weight | 23.0 kg |
| Height | 1.30 m |
| Measurement Information: | |
| Start of measurement | 2010/08/31 14:34:45 |
| Type of measurement | RINT (Respiratory Interruption) |
| Datafile | 100831_Rint_S_20100831_143445.tdms |
| Software-version | v1.11 |
| Cradle | CCMPROTO-10060001 |
| Sensor Used | TSRP-10030001 |
| Sensor Calibration Date | 2010/04/06 |

FIG. 7 shows the results of the Rint-measurement: automatic occlusion of 100 ms after 3 comparable tidal breathings.

FIG. 8 shows the results of the Rint-measurement: zoomed in on the occlusion.

Calculated result for this occlusion:

| | |
|---|---|
| Occlusion | 14:42:02.89 |
| Rrs [kPa * s/l] | 0.2607 |
| Flow [ml/s] | 0.4072 |
| Pint [kPa] | 0.1061 |
| P(t0) [kPa] | 0.1860 |
| Ppre [kPa] | 0.0760 |
| Ppost [kPa] | 0.1821 |
| Slope | 0.3382 |

With reference to FIGS. 9-12 a further development of the measurement device according to the invention will be discussed. The working principle of this measurement device is the same as for the device as disclosed above. The same of similar elements will therefore be indicated with the same reference numerals.

As shown in FIG. 9a, the measurement device generally consists of two pieces: a cradle 12 and a removable flow tube part 17. For releasing the flow tube part 17 from the cradle 12, buttons 20 provided with hooked shaped ends are provided. The hooked shaped ends engage accordingly formed recesses in the cradle 12 in the connected state.

In this example, a flow tube part 17 for SOT-Rint is connected to the cradle 12. The flow tube part 17 is hereto, inter alia, provided with ultrasonic transducers 15 (see FIG. 10) and electronics 16. A mouth piece 18 can be attached to the measurement device such that the mouth piece 18 is in fluid connection with the tube 1, which will be discussed in more detail below. Positioned near the mouthpiece 18 is a connection 21 which cooperates with a pressure sensor in the cradle 12. Also the electrical spring contacts 22 for connecting the electronics 16 to the electronics provided in the cradle 12 are visible in FIG. 9b.

As shown in FIGS. 10, 11 and 12, the flow tube part 17 connected to the lower part of the cradle 12. The flow tube part 12 is provided with parts 22 extending laterally of the tube 1 which form a housing for the transducers 15, thereby sealing the transducers 15. The transducers 15 are arranged to measure the flow in the tube 1. In combination with the measured pressure, air-way resistance can then be calculated during an occlusion of the tube 1.

In this example and with reference to FIG. 12 showing a cross-section of the device in connected state, the flow tube part 17 is provided with a tube 1 having a first length of tube 1b manufactured from a relative stiff material and an end-part 1a manufactured from an elastic material, in this example soft silicon. The tube end 1a is formed by two sheet-like elements 23a and 23b bonded at their lateral edges, thereby forming recesses 3 in the circumference of the tube end 1a.

The cradle 12 is provided with an occlusion mechanism generally indicated with 11. The mechanism 11 comprises an electromotor 10 for rotating a shaft 10a. Connected to this shaft 10a via arms 9a and 9 is the shutter knife 4. Shutter knife 4 is at one end rotably connected with spindle 4a to projecting parts of the cradle 12. In the connected state, the tube 1 extends between the lower ends, and thereby the spindles 4a, of the knife 4.

On actuation of the motor 10, spindle 10a will rotate in a direction indicated with I in FIG. 11 such that knife 4 moves downwardly, indicated with II. The knife 4 hereby engages the tube 1, more in particular upper sheet 23a thereof, forcing element 23a towards lower sheet 23b. Eventually, the flow-path in the tube 1 is closed when the upper sheet 23a lies against lower sheet 23b due to the pressing action of the knife 4. Further movement in direction II will force both sheets 23a and 23b of the tube 1 in a recess 5 (see FIG. 12) provided in a wall section 3 of the housing of the flow tube part 17. This ensures a proper occlusion of the flow-path, see FIG. 1c. Damping members 8 are further provided to limit the movement of the arm 9a, thereby limiting the movement of the knife 4.

Upon disengagement of the motor 10, the knife 4 will return to the position as shown in FIGS. 10 and 11 due to a spring element (not shown). As the tube end 1a is manufactured from an elastic material, the flow-path will open automatically upon disengagement of the knife 4.

The present invention is not limited to the embodiment shown, but extends also to other embodiments falling within the scope of the appended claims.

1. Measurement device for lung function measurement comprising a flow tube forming an air-flow path, an actuator for closing and opening the flow tube and at least one sensor for measuring at least one lung function variable, wherein the actuator is arranged to force the flow tube to close the air-flow path by pressing the tube against a wall of the flow tube.

2. Measurement device according to claim 1, wherein the flow tube has elastic characteristics for opening the air-flow path upon reversal of the actuator.

3. Measurement device according to claim 1, wherein the flow tube comprises a silicon rubber tube.

4. Measurement device according to claim 1, wherein the flow tube is provided with two cut-outs in the contour.

5. Measurement device according to claim 4, wherein the cut-outs are provided at diametrically opposed locations in the contour.

6. Measurement device according to claim 1, wherein the flow tube comprises a stiff wall, wherein the actuator is arranged to force the flow tube to close the air-flow path by pressing the tube against the stiff wall.

7. Measurement device according to claim 6, wherein the wall comprises a U-shaped groove arranged to receive the actuator in the closed position.

8. Measurement device according to claim 1, wherein the actuator is provided with V-shaped end.

9. Measurement device according to claim 1, wherein the actuator is biased towards the open air-flow path position.

10. Measurement device according to claim 1, wherein the sensor is formed integrally and water-tight with the flow tube.

11. Measurement device according to claim 10, wherein the sensor is encapsulated in casing filled with a sealant.

12. Measurement device according to claim 1 formed as a handheld.

13. Measurement device according to claim 1, comprising a cradle and a flow tube part provided with the flow tube as separate bodies, wherein the cradle is arranged to interchangeably receive the flow tube part and wherein the actuator is arranged in the cradle.

14. Measurement device according to claim 13, wherein the flow tube is provided with at least one sensor and wherein the cradle and the flow tube part are provided with cooperating electric spring contacts.

15. Measurement device according to claim 13, wherein the cradle further comprises at least one of a pressure sensor, a battery and digital electronics.

16. Cradle for use in a measurement device according to claim 13.

17. Flow tube or flow tube part for use in a measurement device according to claim 1.
